(19) United States
(12) Patent Application Publication
(10) Pub. No.: US 2026/0066099 A1
(43) Pub. Date: Mar. 5, 2026

(54) METHOD AND SYSTEM FOR QUANTITATIVE MRI USING GENERATIVE AI

(71) Applicant: Siemens Healthineers AG, Forchheim (DE)

(72) Inventors: Mariappan S. Nadar, Plainsboro, NJ (US); Mahmoud Mostapha, Princeton, NJ (US); Radu Miron, Iasi (RO); Boris Mailhe, Plainsboro, NJ (US); Nirmal Janardhanan, Monmouth Junction, NJ (US); Weijie Gan, St. Louis, MO (US); Marcel Dominik Nickel, Herzogenaurach (DE); Thorsten Feiweier, Poxdorf (DE); Rainer Schneider, Höchstadt (DE); David Grodzki, Erlangen (DE); Omar Darwish, London (GB); Tobias Würfl, Erlangen (DE); Till Hülnhagen, Erlangen (DE); Jens Gühring, Erlangen (DE)

(21) Appl. No.: 19/016,512

(22) Filed: Jan. 10, 2025

Related U.S. Application Data
(60) Provisional application No. 63/687,806, filed on Aug. 28, 2024.

(30) Foreign Application Priority Data
Aug. 28, 2024 (EP) 24465562.7

(51) Int. Cl. G16H 30/40; G06T 11/00

(52) U.S. Cl. CPC G16H 30/40; G06T 12/20; G06T 2211/441

(57) ABSTRACT

Systems and methods for image reconstruction and quantitative MRI. Generative models such as diffusion models are used to reconstruct MR images and generative models and constrained mathematical models fit to estimate quantitative maps from the reconstructed MR images.

### CROSS REFERENCE TO RELATED APPLICATIONS

This application claims the benefit of the filing date under 35 U.S.C. § 119(e) of Provisional U.S. Patent Application Ser. No. 63/687,806, filed Aug. 28, 2024 and European Patent Application No. 24465562.7, filed Aug. 28, 2024, both of which are hereby incorporated by reference.

### FIELD

This disclosure relates to medical imaging.

### BACKGROUND

Magnetic resonance imaging, or MRI, is a noninvasive medical imaging test that can generate detailed images of almost every internal structure in the human body, including, for example organs, bones, muscles, and blood vessels. Traditionally, contrast-weighted MR images are interpreted visually, while the absolute images' intensity is arbitrary. Quantitative MRI (qMRI) provide an objective and efficient way to represent certain data in the MR data, where numeric values of the tissue properties are measured. The ensuing quantitative markers may be used to probe both macro- and microscopic information while optimally utilizing the dynamic range of MRI contrast mechanisms. However, qMRI requires the acquisition/measurement of several high SNR (signal to noise ratio) MR images, which may be a very time-consuming process. These multiple high SNR scanned MR images may be necessary for fitting advanced physical/mathematical models for quantitative tissue properties. However, it is also important to reduce the acquisition time significantly to overcome numerous problems arising from very long scan times.

Accelerating the acquisition/scanning of MR images have been addressed over the years using technologies such as parallel imaging, compressed sensing, unrolled deep learning (DL) solutions with good success. In addition, unrolled DL solutions may use deep regression networks for regularizers for scanned MR weighted images. In these procedures, the quantitative tissue parametric maps have typically been computed by a standard least square fitting of appropriate physical models using the scanned MR images as samples for the fit. However, this approach is fraught with problems: long acquisition times for the scanned MR weighted images, less than ideal reconstructions of the scanned MR images, and approximate model fitting resulting in estimated quantitative tissue properties map that are traditionally un-regularized.

### SUMMARY

By way of introduction, the preferred embodiments described below include methods, systems, instructions, and/or computer readable media for image reconstruction and quantitative maps using generative models.

In a first aspect, a method for quantitative MRI using generative AI is provided; the method comprising: training a first generative model to reconstruct an MR image from acquired MR data; and training a second generative model to generate quantitative map data from the acquired MR data, wherein a data consistency term derived from the acquired MR data is used for regularization of the second generative model; and storing the first generative model and second generative model.

In a second aspect, a method for quantitative MRI is provided, the method comprising: acquiring MR imaging data; inputting the MR imaging data into a first generative model trained to reconstruct an image and a second generative model trained to generate quantitative MRI data, wherein the first generative model is constrained by a data consistency term based on measurement data, wherein the second generative model is regularized by a constrained mathematical model fit, and outputting the reconstructed image and the quantitative MRI data.

In a third aspect, a system for quantitative MRI is provided, the system comprising: a medical imaging device configured to acquire MR data; a memory configured to store a first generative model configured to reconstruct an MR image from the MR data and a second generative model trained to learn a probability density of quantitative map data and generate quantitative map data wherein the quantitative map data generation is constrained by an exponential fit provided by a priori probability density function from the first generative model; and a processor configured to reconstruct an MR image using the first generative model and generate a quantitative map using the second generative model.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 depicts an example system for magnetic resonance imaging and generation of quantitative maps according to an embodiment.

FIG. 2 depicts an example workflow of a diffusion learning process according to an embodiment.

FIG. 3 depicts several examples of hallucinations generated by generative AI processes.

FIG. 4 depicts an example workflow for a denoising diffusion probabilistic model (DDPM) for Plug-N-Play (PnP) Image Reconstruction of MRI data according to an embodiment.

FIG. 5 depicts an example algorithm for a denoising diffusion probabilistic model (DDPM) for Plug-N-Play (PnP) Image Reconstruction of MRI data according to an embodiment.

FIG. 6 depicts an example method for magnetic resonance imaging and generation of quantitative maps according to an embodiment.

FIG. 7 depicts an example workflow for magnetic resonance imaging and generation of quantitative maps according to an embodiment.

FIG. 8 depicts an example U-net architecture for magnetic resonance imaging and generation of quantitative maps according to an embodiment.

FIG. 9 depicts an example method for magnetic resonance imaging and generation of quantitative maps according to an embodiment.

FIG. 10 depicts an example workflow for magnetic resonance imaging and generation of quantitative maps according to an embodiment.

FIG. 11 depicts an example method for magnetic resonance imaging and generation of quantitative maps according to an embodiment.

FIG. 12 depicts an example workflow for magnetic resonance imaging and generation of quantitative maps according to an embodiment.

FIG. 13 depicts an example system for magnetic resonance imaging and generation of quantitative maps according to an embodiment.

FIG. 14 depicts an example artificial neural network according to an embodiment.

FIG. 15 depicts an example convolution neural network according to an embodiment.

### DETAILED DESCRIPTION

Embodiments described herein provide systems and methods that use generative models (e.g. diffusion models) and constrained physical/statical models to reconstruct contrast weighted MR images and generative models and constrained mathematical models fit to estimate quantitative maps (that captures a specific tissue property) from reconstructed contrast weighted MR images.

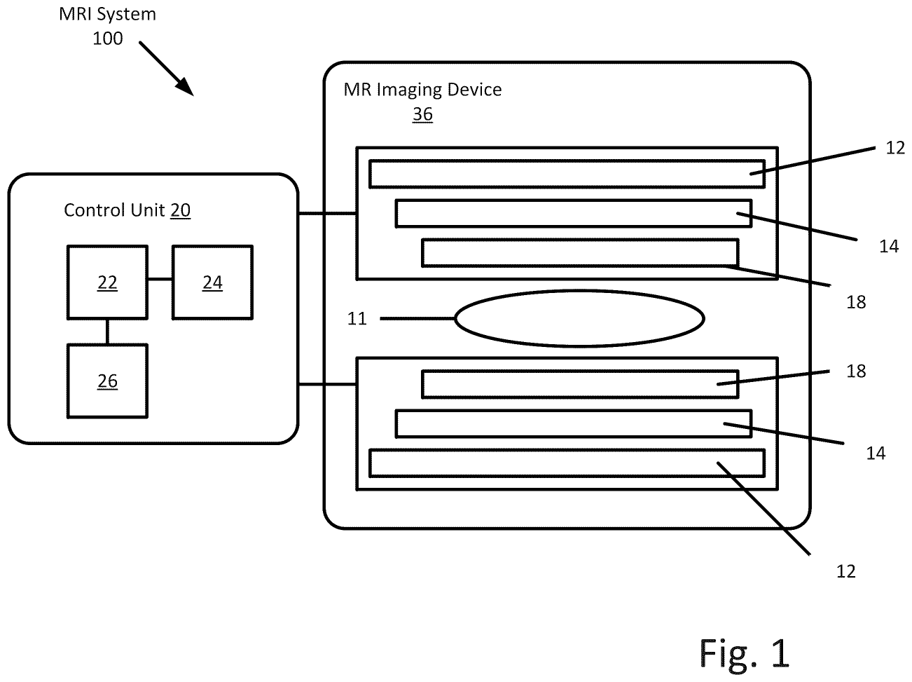

FIG. 1 depicts an example system 100 for magnetic resonance imaging and generation of quantitative maps. MRI is a noninvasive medical imaging procedure that can generate detailed images of internal structures in the human body, for example, organs, bones, muscles, and blood vessels. Quantitative MRI further includes the generation of data or maps of physical or chemical variables that are measured in physical units and compared between tissue regions and among subjects.

The examples described herein use a magnetic resonance (MR) context (i.e., a magnetic resonance scanner), but the reconstruction techniques, quantitative maps, and generative models may be used for other medical imaging procedures such as computed tomography (CT) or positron emission tomography (PET) where applicable. The examples further use knee and brain MRI procedures as an example, but any organ or region may be imaged by the system 100. The system 100 uses generative model(s) to provide the MR contrast weighted image and quantitative maps that capture a specific tissue property. In this example, MRI data is acquired by the MR system 100. The MR system 100 includes an MR scanner 36 or system, a computer based on data obtained by MR scanning, a server, or another processor 22. The MR imaging device 36 is only exemplary, and a variety of MR scanning systems may be used to collect the MR data. The MR imaging device 36 (also referred to as a MR scanner or image scanner) is configured to scan a patient 11. The scan provides scan data in a scan domain. The MR imaging device 36 scans a patient 11 to provide k-space measurements (measurements in the frequency domain). The MR system 100 further includes a control unit 20 configured to process the MR signals and generate (reconstruct) images of the object or patient 11 for display to an operator or further analysis. The control unit 20 includes a processor 22 that is configured to execute instructions, or the method described herein. The control unit 20 may store the MR signals and images in a memory 24 for later processing or viewing. The control unit 20 may include a display 26 for presentation of images to an operator.

In the MR system 100, magnetic coils 12 create a static base or main magnetic field B0 in the body of patient 11 or an object positioned on a table and imaged. Within the magnet system are gradient coils 14 for producing position dependent magnetic field gradients superimposed on the static magnetic field. Gradient coils 14, in response to gradient signals supplied thereto by a gradient and control unit 20, produce position dependent and shimmed magnetic field gradients in three orthogonal directions and generate magnetic field pulse sequences. The shimmed gradients compensate for inhomogeneity and variability in an MR imaging device magnetic field resulting from patient anatomical variation and other sources. The control unit 20 may include a RF (radio frequency) module that provides RF pulse signals to RF coil 18. The RF coil 18 produces magnetic field pulses that rotate the spins of the protons in the imaged body of the patient 11 by ninety degrees or by one hundred and eighty degrees for so-called “spin echo” imaging, or by angles less than or equal to 90 degrees for “gradient echo” imaging. Gradient and shim coil control modules in conjunction with RF module, as directed by control unit 20, control slice-selection, phase-encoding, readout gradient magnetic fields, radio frequency transmission, and magnetic resonance signal detection, to acquire magnetic resonance signals representing planar slices of the patient 11. In response to applied RF pulse signals, the RF coil 18 receives MR signals, e.g., signals from the excited protons within the body as the protons return to an equilibrium position established by the static and gradient magnetic fields. The MR signals are detected and processed by a detector within RF module and the control unit 20 to provide an MR dataset to a processor 22 for processing into an image. In some embodiments, the processor 22 is located in the control unit 20, in other embodiments, the processor 22 is located remotely. A two or three-dimensional k-space storage array of individual data elements in a memory 24 of the control unit 20 stores corresponding individual frequency components including an MR dataset. The k-space array of individual data elements includes a designated center, and individual data elements individually include a radius to the designated center. The magnetic field generator (including coils 12, 14 and 18) generates a magnetic field for use in acquiring multiple individual frequency components corresponding to individual data elements in the storage array. A storage processor in the control unit 20 stores individual frequency components acquired using the magnetic field in corresponding individual data elements in the array. The row and/or column of corresponding individual data elements alternately increases and decreases as multiple sequential individual frequency components are acquired. The magnetic field generator acquires individual frequency components in an order corresponding to a sequence of substantially adjacent individual data elements in the array, and magnetic field gradient change between successively acquired frequency components is substantially minimized.

When applied, the MR imaging device 36 is configured by the imaging protocol to scan a region of a patient 11. For example, in MR, such protocols for scanning a patient 11 for a given examination or appointment include diffusion-weighted imaging (acquisition of multiple b-values, averages, and/or diffusion directions), turbo-spin-echo imaging (acquisition of multiple averages), and/or contrast. In one embodiment, the protocol is for compressed sensing. The control unit 20 is configured to reconstruct an image using the acquired MRI data from an imaging procedure. Image reconstruction may be performed by the system 100 or other computing devices.

In embodiments described herein, image reconstruction uses a generative deep learning framework for generating images. The generative deep learning models, such as diffusion models, utilize prior knowledge either with (supervised) or without (unsupervised) knowledge of a specific reconstruction task. By decoupling learning of the prior knowledge from the reconstruction task, the diffusion models may overcome existing issues of costly training and poor robustness to varied scan parameters.

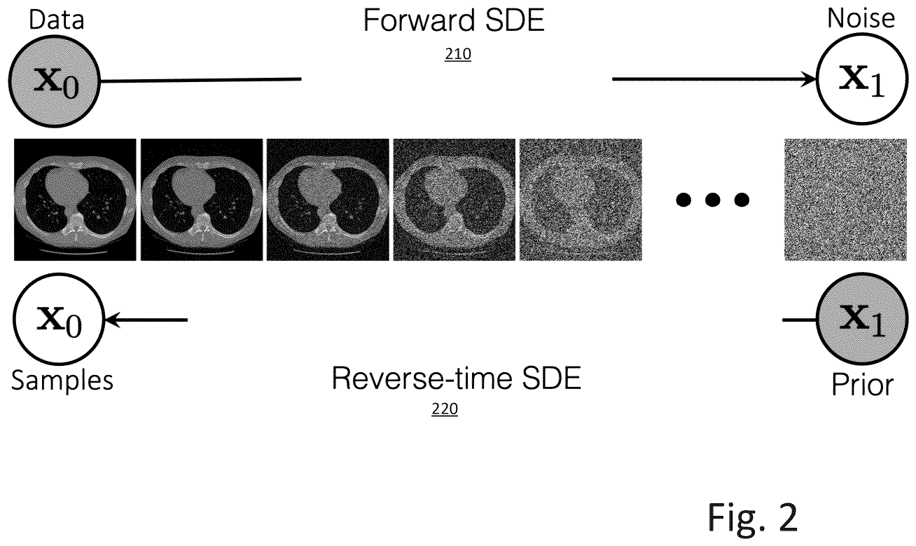

FIG. 2 depicts an example of a generative process including a forward process 210 and reverse process 220 also referred to as the inference stage. The goal of diffusion models is to learn a diffusion process for a given dataset, such that the process can generate new elements that are distributed similarly as the original dataset. In the forward stochastic differential equation (SDE) noise is added to the input image over and over again until the image is practically all noise. At each step, the model learns how to map images to their corresponding noise-free measurements. In the reverse step, the learned model is used to recover the data by reversing this noising process. Image reconstruction in MRI is a similar inverse problem that attempts to find an image from noisy scan measurements. To solve the inverse problem a forward model is defined that maps noisy MR images to their corresponding noise-free measurements. As measurements become noisier (for example as scan time is reduced) or less complete (for example when using increased acceleration), the resulting image reconstruction problem becomes highly ill-posed, meaning it has no stable, unique solution. In such situations the acquired measurements are said to be sparse, i.e., they are generally insufficient to uniquely specify a finite-dimensional approximation of the sought-after object, even in the absence of measurement noise or errors related to modeling the imaging system. False structures may arise due to the reconstruction method incorrectly estimating parts of the object that either did not contribute to the observed measurement data or cannot be recovered in a stable manner, a phenomenon that is referred to as hallucinations.

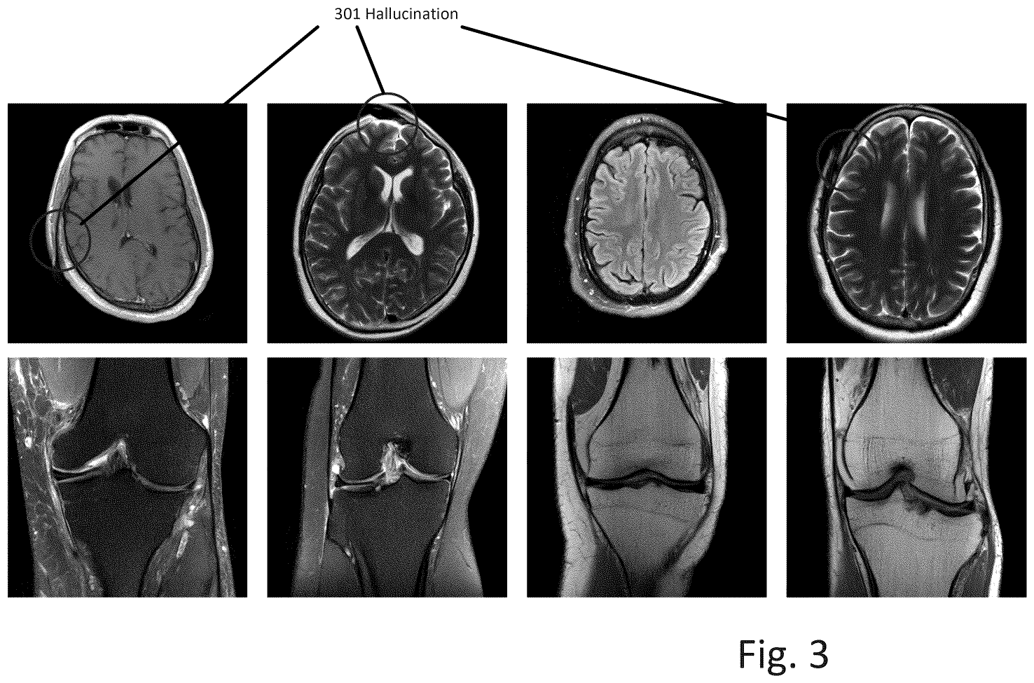

FIG. 3 depicts various hallucinations 301 in MRI images. For example, in the brain MRI images, the bone structure is poorly generating leading to gaps in the structure. While these errors are obvious, less pronounced hallucinations may lead to poor diagnostics or analysis where it may be difficult to determine if a feature is an actual feature or a hallucination. Hallucinations may be resolved by incorporating information about the distribution of probable images, so-called prior knowledge. The reconstructed image balances maximizing both the likelihood that explains measurements, and the prior, that is, the probability that is a valid medical image. In inverse problems in medical imaging, the generative models (for e.g. diffusion models) capture rich image priors from underlying data distributions. From a Bayesian perspective, the diffusion models learn the a priori probability density function of the images. Solving the Bayesian inverse problem is tantamount to drawing posterior samples (and/or computing the posterior mean) from the posterior density function that is a product of the likelihood function (physical and statistical model of the imaging system) and the learnt a priori probability density function.

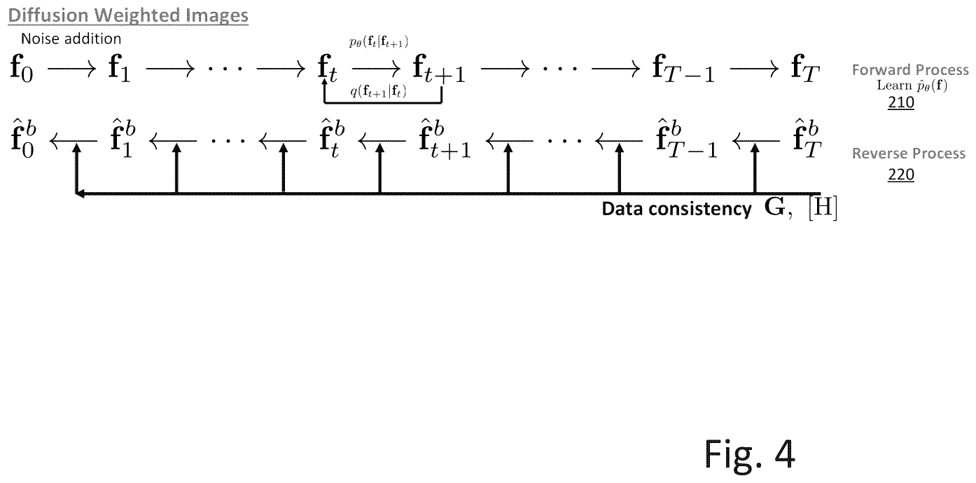

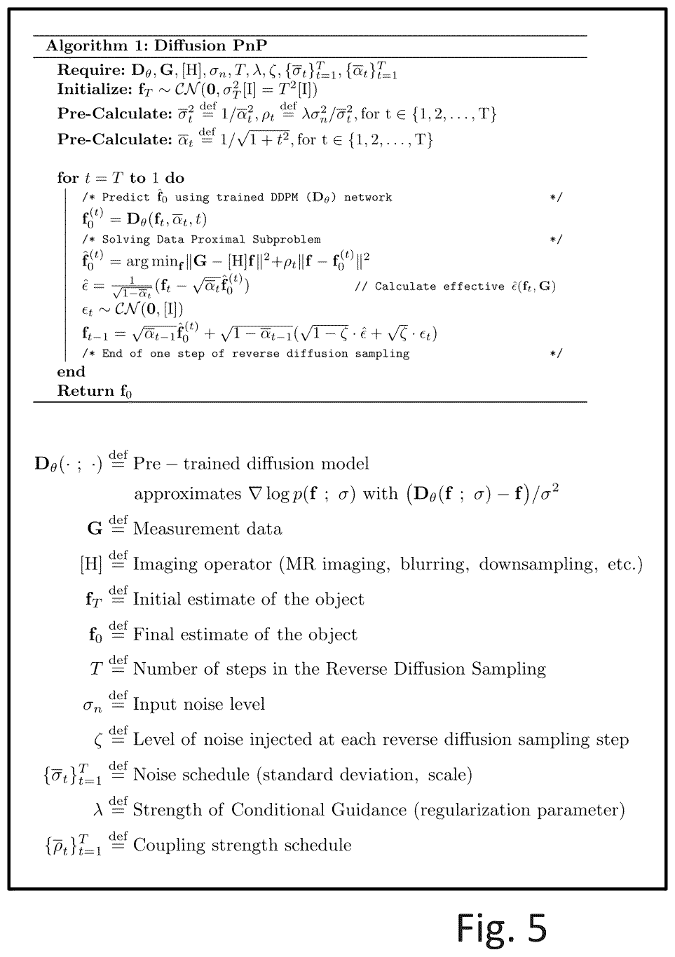

FIG. 4 depicts an example denoising diffusion probabilistic model (DDPM) for Plug-N-Play (PnP) Image Reconstruction of MRI data, e.g., the first generative model 400. FIG. 5 depicts an algorithm (Diffusion PnP) and the constants/variables for the Denoising Diffusion Probabilistic Model of FIG. 4. PnP typically implies replacing a proximal operator in an iterative algorithm with a state-of-the-art image denoiser. In FIG. 4, the forward process 210 learns the probability density function of contrast weighted MR image data by adding noise to the input image data. In the reverse process 210, an image is generated using the learned probability density function of contrast weighted MR image data while being constrained by a data consistency term G that represents expected/known measurements.

In FIG. 5, a method/algorithm is described that combines the traditional plug-and-play method with the diffusion sampling framework to accurately restore complex MRI data regarding reconstruction faithfulness and perceptual quality. The diffusion model includes measurement during reverse diffusion steps, which is based on denoising diffusion implicit models (DDIM) and supports fast sampling. This measurement is carried out after a correction step that accounts for the inaccurate estimation resulting from computing the proximal solution. As depicted in FIG. 4, the diffusion process is split into forward and reverse diffusion processes. The forward diffusion process is a process of turning an image into noise, and the reverse diffusion process is supposed to turn that noise into the image again. The reverse process 210 starts with a noisy image. The process continuously denoises the image over and over again to steer it in a particular direction. The value T describes how many inference steps will be taken during this process. The higher the value, the more steps that are taken to produce the image (also more time). The goal of the reverse diffusion process is to convert a noisy image (for example acquired using sparse data from an MRI procedure) into an cleaner/higher resolution image.

In FIGS. 4 and 5, data consistency is performed during the inference steps using measurement data G. The data consistency step steers the image reconstruction in a particular direction. The output of the image reconstruction is a contrast weighted image. In addition to MRI images, there are also several relevant quantitative imaging biomarkers that can be derived from specific MRI techniques. One such technique is diffusion-weighted MRI (DW-MRI). DW MRI creates images based on water molecule diffusion. The extent of tissue cellularity and the presence of intact cell membrane help determine the impedance of water molecule diffusion. This impedance of water molecules diffusion may be quantitatively assessed using an apparent diffusion coefficient (ADC) value. In embodiments described below, the ADC value is used as an example quantitative imaging biomarker that is derived from DW-MRI data. The ADC may be used to stage tumors, assess treatment response, and predict tumor aggressiveness among other uses. The ADC is typically calculated by applying at least two different strengths of gradients (denoted as b-values), allowing for the measurement of molecular diffusion independent of other MRI signal influences like the T2 shine-through effect. The resulting ADC value is inversely proportional to the degree of diffusion restriction: higher ADC values indicate freer water mobility (typical of fluids or necrotic tissue), whereas lower ADC values suggest restricted diffusion, as seen in dense cellular structures or fibrotic tissue. This quantitative measurement provides critical insights into the microscopic structure and pathology of tissues. While the examples below describe the generation of ADC maps, the ADC is just one quantitative measurement among many that may be provided by Quantitative MRI. Quantitative MRI (qMRI) may be used to measure a wide range of MR properties such as ADC, T1, T2, and T2* relaxation times; proton-density (PD); magnetization transfer ratio (MTR), inhomogeneous MTR, MT saturation (MTsat); quantitative susceptibility maps (QSM); mean diffusivity (MD); fractional anisotropy (FA); water fraction (WF); and macromolecular tissue volume fraction (MTVF) among others. Similar mechanisms as described below for computing the ADC maps may be used for these and other quantitative maps/values.

Traditional generation of quantitative maps such as for ADC typically involves reconstructing contrast weighted MR images and performing a standard least squares fit to a mathematical model. Typically, no regularization (or a priori information) is injected in this process. This approach is fraught with problems, including for example long acquisition times for the scanned MR weighted images, less than ideal reconstructions of the scanned MR images, and approximate model fitting resulting in estimated quantitative tissue properties map that are traditionally un-regularized. Embodiments provide a Bayesian approach where the a priori probability density function is a trained generative model that captures a rich representation of the quantitative map. This enables performance and efficiency improvements compared to traditional approaches due to the rich a priori information obtained via training samples.

Embodiments described herein provide generative models (e.g. diffusion models) and constrained physical/statical models to reconstruct contrast weighted MR images and generative models and a constrained mathematical model fit to estimate quantitative maps (that captures a specific tissue property) from reconstructed contrast weighted MR data. In a first embodiment, a sequence of contrast weighted MR image data and quantitative map data are learned (i.e. probability density function of contrast weighted MR image data and probability density function of quantitative map data, i.e. proxies such as score function of the corresponding probability density functions) and inferred separately. In a second embodiment, separate training is used for learning contrast weighted MR image data and quantitative map data (i.e. probability density function of contrast weighted MR image data and probability density function of quantitative map data, i.e. proxies such as score function of the corresponding probability density functions), but joint consideration is used during inference. In a third embodiment, joint learning of contrast weighted MR image data and quantitative maps (i.e. joint probability density function of contrast weighted MR image data and quantitative map data, i.e. proxy such as score function of joint probability density function) and inference steps is used. As noted above, these approaches may be applied to different quantitative MR imaging problems in general. Examples include but are not limited to: Diffusion-related parameters (for example ADC, tensor parameters (FA, RA, . . . ), IVIM parameters, Kurtosis parameters, T1, T2, T2*, T1r, Muscle fat/iron, Volumetry (no fitting), e.g. brain, cardiac, Perfusion (brain, cardiac): blood flow, blood volume, time-to-peak, mean transit time, Flow, Tissue stiffness (elastography), Dynamic contrast enhancement DCE, Quantitative susceptibility mapping QSM (quite complex dipole models), Chemical exchange saturation transfer CEST, Magnetization transfer/transfer ratio, Spectroscopy, Temperature mapping etc.

A diffusion model is used as a primary example, however other generative models may include but are not limited to Auto Encoders, Variational Auto Encoders (VAE), Denoising Auto Encoders, Restricted Boltzmann Machine (RBM), Generative Adversarial Networks (GAN), Denoising Diffusion Probabilistic Models (DDPM), Score-based Diffusion Models, Poisson Flow Generative Models (PFGM and PFGM++), Flow Matching, Rectified Flow, Auto Regressive (AR) models, etc. For example, Generative adversarial networks may be used to generate new data. For example, based on a set of MRI images, a GAN may generate synthetic images that look at least superficially authentic to human observers, and may also be used as synthetic training data for other machine learning models.

The generative adversarial model includes a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

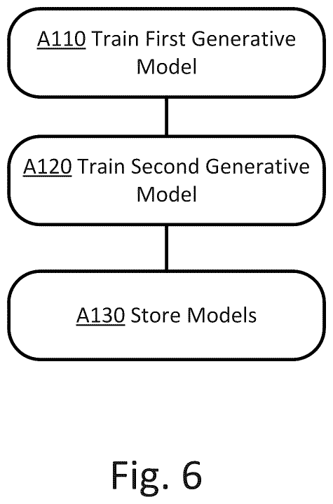

FIG. 6 depicts an example method for quantitative MRI using generative AI where a sequence of contrast weighted MR image data and quantitative map data are learned (i.e. probability density function of contrast weighted MR image data and probability density function of quantitative map data, i.e. proxies such as score function of the corresponding probability density functions) and inferred separately.

At act A110, a first generative model 400 to estimate a MR image is trained using a priori information comprising measurement data for the particular type of scan/region. In an embodiment, the model is a generative model, in particular a diffusion model, for example, a denoising diffusion probabilistic model. FIGS. 4 and 5 described above depict an example of a training mechanism for providing a model to estimate a contrast weighted MR image. In the learning phase, the forward process 210 learns the probability density function of contrast weighted MR image data by adding noise to the input image data. In the reverse process 210, an image is synthesized using the learned probability density function of contrast weighted MR image data. Unlike standard diffusion models, a data consistency term G is used. G may include measurements/linear transform of known features of the region or object being scanned. In an embodiment, a regularization term may be included such as subspace approaches, MP, PCA etc. on the sequence of contrast weighted MR images.

At act A120, a second generative model for estimating the quantitative map data is trained sequentially with the first generative model 400 wherein a data consistency term derived from the MR image data of the first generative model 400 is used for regularization. In an embodiment the second generative model is a diffusion model, for example, a denoising diffusion probabilistic model (DDPM) or a denoising diffusion implicit model (DDIM). In an embodiment, the quantitative map data is for ADC. Apparent Diffusion Coefficient (ADC) is a quantitative measurement of water molecule diffusion within tissue that is calculated using magnetic resonance imaging (MRI) with diffusion-weighted imaging (DWI). In DWI, magnetic gradients are applied to cause dephasing of spins in moving water molecules, leading to a loss of signal from areas of higher molecular motion. The ADC is calculated by applying at least two different strengths of these gradients (denoted as b-values). These b-values are used in the training process of the second generative model for regularization, for example by fitting a mathematical exponential curve to the signal intensity data acquired at different b-values on a diffusion-weighted imaging (DWI) sequence. In the forward stage of the training process, the model starts with an ADC map and adds noise to it in small steps, making it gradually more and more distorted. This creates a series of distorted ADC maps, each one being a little more distorted than the previous one. In the reverse stage, the model learns to reverse the forward process 210. It takes a distorted ADC map and removes the noise step by step to recover the original ADC map. The model does this by learning from a large number of examples of distorted maps and their original versions. Once the model has learned how to reverse the noise-adding process, it can generate new ADC maps. The reverse process 210 starts with a completely distorted map (random noise) and removes the noise step by step to create a new, clear ADC map. Since the model has learned the reverse process 210 from real ADC maps, the new ADC map that is generated look similar to an actual ADC map. The generation process is further constrained by B-values from input MR data.

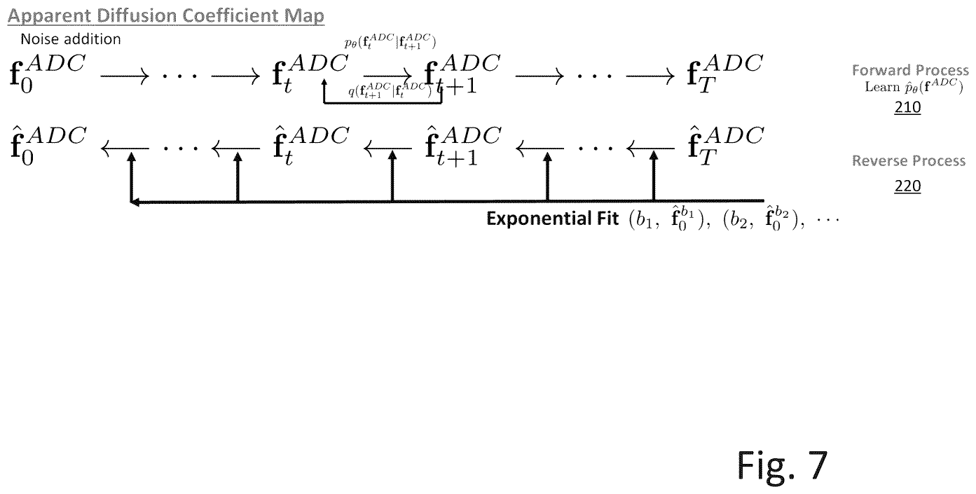

FIG. 7 depicts an example workflow for training the second generative model 700. The first generative model 400 may be trained as described in FIGS. 4 and 5. The second generative model 700 is similarly trained using a diffusion process in an attempt to learn the probability density function of the quantitative map data by adding noise. An exponential fit is used in the reverse process 210 to constrain the output. Here, for the ADC, the B values provided by the first model are used for regularization.

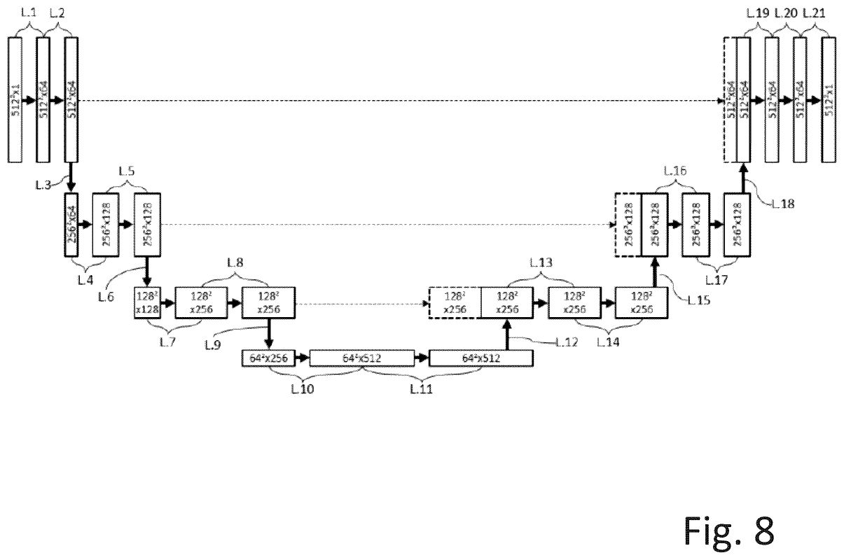

Different training mechanisms may be used, such as reparameterization or score-based generative modeling. In other embodiment, different types of generative AI models may be used for the first generative model 400 or second generative model 700 such as Auto Encoders, Variational Auto Encoders (VAE), Denoising Auto Encoders, Restricted Boltzmann Machine (RBM), Generative Adversarial Networks (GAN), Denoising Diffusion Probabilistic Models (DDPM), Score-based Diffusion Models, Poisson Flow Generative Models (PFGM and PFGM++), Flow Matching, Rectified Flow, Auto Regressive (AR) models, etc. In an embodiment, the first generative model 400 and second generative model 700 are based on is a convolutional neural network, in particular, a convolutional neural network having a U-net structure, for example as displayed in FIG. 8. The input data to the machine learning network is a two-dimensional medical image comprising 512×512 pixel, every pixel comprising one intensity value (e.g., relating to the Hounsfield units of the respective pixels). The machine learning network comprises convolutional layers (indicated by solid, horizontal arrows), pooling layers (indicating by solid arrows pointing down), and upsampling layers (indicated by solid arrows pointing up), the number of the respective nodes is indicated within the boxes. Within the U-net structure first the input images are downsampled (decreasing the size of the images and increasing the number of channels), afterwards they are upsampled (increasing the size of the images and decreasing the number of channels) to generate a transformed image.

All except the last convolutional layers L.1, L.2, L.4, L.5, L.7, L.8, L.10, L. 11, L.13, L. 14, L.16, L. 17, L. 19, L.20 use 3×3 kernels with a padding of 1, the ReLU activation function, and a number of filters/convolutional kernels that matches the number of channels of the respective node layers as indicated in FIG. 8. The last convolutional layer uses a 1x 1 kernel with no padding and the ReLU activation function.

The pooling layers L.3, L.6, L.9 are max-pooling layers, replacing four neighboring nodes with only one node, the value being the maximum of the values of the four neighboring nodes. The upsampling layers L.12, L.15, L.18 are transposed convolution layers with 3×3 kernels and stride 2, which effectively quadruple the number of nodes. The dashed horizontal errors correspond to concatenation operations, where the output of a convolutional layer L.2, L.5, L.8 of the downsampling branch of the U-net structure is used as additional inputs for a convolutional layer L.13, L.16, L.19 of the upsampling branch of the U-net structure. This additional input data is treated as additional channels in the input node layer for the convolutional layer L.13, L.16, L.19 of the upsampling branch.

At act A130, the trained models for generating the MR image and the quantitative map data is output. The models may be applied to newly acquired MRI data in order to generate MR image data and quantitative maps.

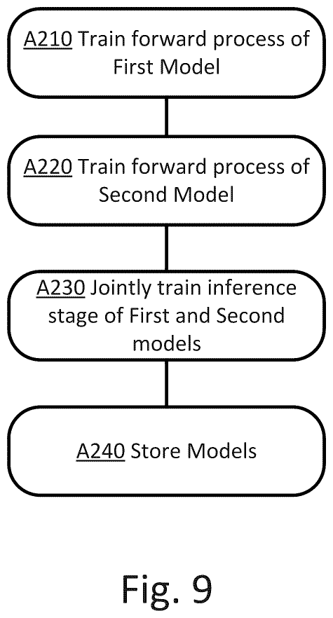

FIG. 9 depicts another example method for quantitative MRI using generative AI where separate training is used for learning the contrast weighted MR image data and the quantitative map data (i.e. probability density function of contrast weighted MR image data and probability density function of quantitative map data, i.e. proxies such as score function of the corresponding probability density functions), but joint consideration is used during inference. The first generative model 400 and second generative model 700 may be configured as described above in FIGS. 4, 5, and 8. For example, the model may be diffusion models based on a U-net structure. The training of the models however differs from the training of the models in FIG. 6 above as joint consideration is used during the reverse phase.

At act A210, a first generative model 400 is trained to estimate a MR image using a priori information comprising measurement data for the particular type of scan/region. In an embodiment, the model is a generative model, in particular a diffusion model, for example, a denoising diffusion probabilistic model. FIGS. 4 and 5 described above depict an example of a training mechanism for providing a model to estimate a contrast weighted MR image. In the learning phase, the forward process 210 learns the probability density function of contrast weighted MR image data by adding noise to the input image data.

At act A220, a second generative model 700 for estimating the quantitative map data is trained sequentially with the first generative model 400. In an embodiment the second generative model 700 is a diffusion model, for example, a denoising diffusion probabilistic model (DDPM) or a denoising diffusion implicit model (DDIM). In an embodiment, the quantitative map data is for ADC. Apparent Diffusion Coefficient (ADC) is a quantitative measurement of water molecule diffusion within tissue that is calculated using magnetic resonance imaging (MRI) with diffusion-weighted imaging (DWI). In DWI, magnetic gradients are applied to cause dephasing of spins in moving water molecules, leading to a loss of signal from areas of higher molecular motion. The ADC is calculated by applying at least two different strengths of these gradients (denoted as b-values). These b-values are used in the training process at act A230 below of the second generative model 700 for regularization, for example by fitting a mathematical exponential curve to the signal intensity data acquired at different b-values on a diffusion-weighted imaging (DWI) sequence. Instead of performing the reverse process 210 and regularization steps independently from that of the first generative model 400, the inference steps are jointly learned.

In the forward stage of the training process, the model starts with an ADC map and adds noise to it in small steps, making it gradually more and more distorted. This creates a series of distorted ADC maps, each one being a little more distorted than the previous one.

At act A230, the inference stage of the first model and second model are jointly trained. In the reverse stage, the first generative model 400 and the second generative model 700 learn to reverse the forward process 210. For example, the second generative model 700 takes a distorted ADC map and removes the noise step by step to recover the original ADC map. The model does this by learning from a large number of examples of distorted maps and their original versions. Once the model has learned how to reverse the noise-adding process, it can generate new ADC maps. The reverse process 210 starts with a completely distorted map (random noise) and removes the noise step by step to create a new, clear ADC map. Since the model has learned the reverse process 210 from real ADC maps, the new ADC map that is generated look similar to an actual ADC map.

In the reverse process 210, an image is synthesized using the learned probability density function of contrast weighted MR image data. Unlike standard diffusion models, a data consistency term G is used. G may include measurements/linear transform of known features of the region or object being scanned. A priori information is used for inference for the first generative model 400 and a data consistency term (B value) derived from the MR image data of the first generative model 400 is used for regularization of the second generative model 700. At act A240, the trained models for generating the MR image and the quantitative map data is stored/output.

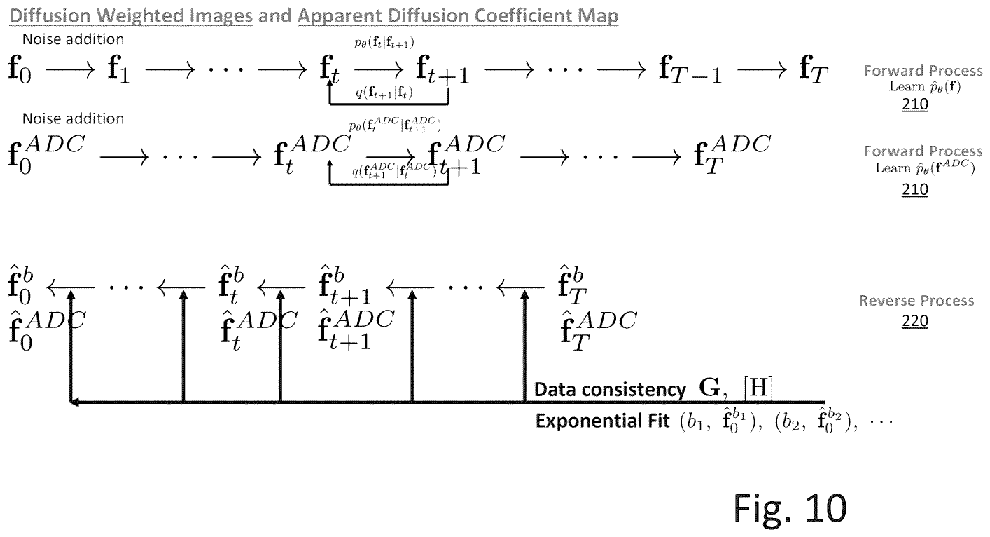

FIG. 10 depicts an example workflow for training the first generative model 400 and second generative model 700 according to the process of FIG. 9. As depicted the forward processes are trained separately but the reverse process is trained jointly.

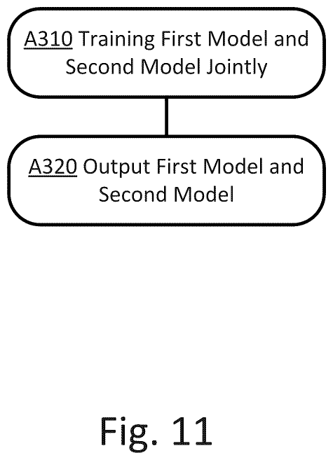

FIG. 11 depicts another example method for quantitative MRI using generative AI where joint training of the contrast weighted MR image data and quantitative maps (i.e. joint probability density function of contrast weighted MR image data and quantitative map data, i.e. proxy such as score function of joint probability density function) and inference steps is used. The first generative model 400 and second generative model 700 may be configured as described above in FIGS. 4, 5, and 8. For example, the model may be diffusion models based on a U-net structure. The training of the models however differs from the training of the models in FIG. 6 above as the models are jointly trained. The training of FIG. 11 may be more computationally complex and may require additional resources than the workflows of FIGS. 6 and 9. The models of FIG. 11, however, may be more accurate.

At act A310, a first generative model 400 to estimate a MR image is trained jointly with a second generative model 700 for estimating the quantitative map data. A priori information is used for inference for the first generative model 400 and a data consistency term (B value) derived from the MR image data of the first generative model 400 is used for regularization of the second generative model 700. The joint training of the two models may be more computationally expensive than training the models separately but may provide more accurate outputs as the reconstructed image and quantitative maps are related. At act A320, the trained models for generating the MR image and the quantitative map data are stores/output.

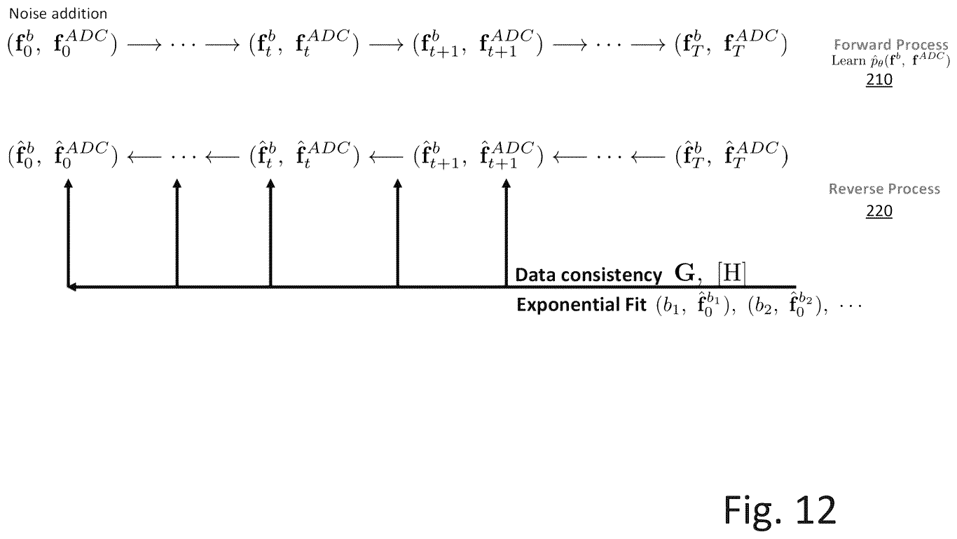

FIG. 12 depicts an example workflow for training the first generative model 400 and second generative model 700 according to the process of FIG. 11. As depicted the forward processes and the reverse process are trained jointly.

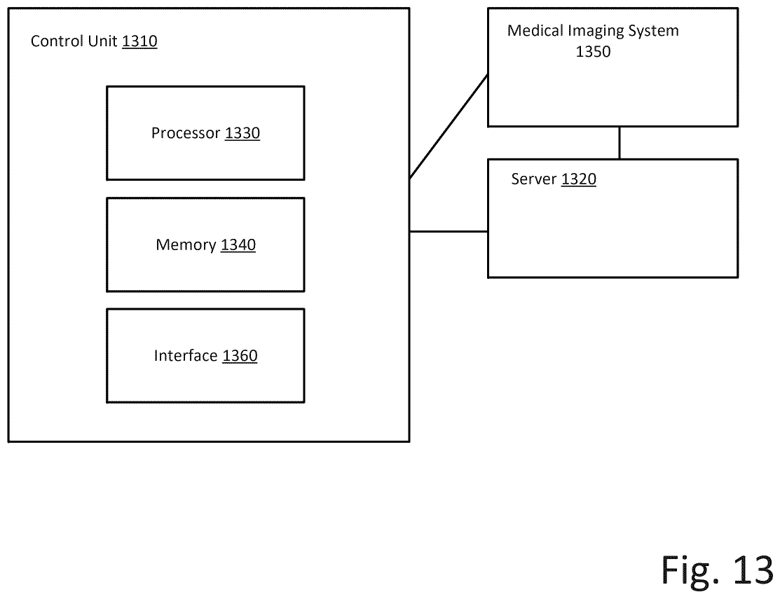

FIG. 13 depicts an example system for quantitative MRI using generative AI. The system includes a medical imaging device 1350, a server 1320, and a control unit 1310 comprising a processor 1330, a memory 1340, and an interface 1360. The medical imaging device 1350 is configured to acquire MR imaging data. The processor 1330 is configured to implement models configured to output MR images and quantitative maps when input the MR imaging data. The memory 1340 is configured to store instructions and the parameters for the model(s). The interface 1360 is configured to display the MR images and quantitative maps and/or accept inputs from a user. The sever 1320 may perform similar tasks as the control unit 1310 and/or may provide some additional processing, storage, or analysis for example using a cloud based platform.

In an embodiment, the medical imaging device 1350 is an MR imaging device 100, for example, as described above in FIG. 1. The MR system 100 of FIG. 1 includes an MR scanner 36 or system, a computer based on data obtained by MR scanning, a server, or another processor 22. The MR imaging device 36 is only exemplary, and a variety of MR scanning systems may be used to collect the MR data. The MR imaging device 36 (also referred to as a MR scanner or image scanner) is configured to scan a patient 11. The scan provides scan data in a scan domain. The MR imaging device 36 scans a patient 11 to provide k-space measurements (measurements in the frequency domain).

The processor 1330 may include an image processor that generates images and quantitative maps using a machine learning network (machine learning model). The processor 1330 may be the processor 22 of FIG. 1. The image processor is a general processor, digital signal processor, three-dimensional data processor, graphics processing unit, application specific integrated circuit, field programmable gate array, artificial intelligence processor, digital circuit, analog circuit, combinations thereof, or another now known or later developed device for image generation. The image processor is a single device, a plurality of devices, or a network. For more than one device, parallel or sequential division of processing may be used. Different devices making up the image processor may perform different functions. In one embodiment, the image processor is also a control processor or other processor of the imaging device. Other image processors of the imaging device or external to the imaging device may be used. The image processor is configured by software, firmware, and/or hardware to process the data acquired by the imaging device and output one or more images and quantitative map data.

The instructions for implementing the processes, methods, and/or techniques discussed herein are provided on non-transitory computer-readable storage media or memories, such as a cache, buffer, RAM, removable media, hard drive, or other computer readable storage media for example the memory 1340. The instructions are executable by the processor or another processor. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, processor or processing strategy and may be performed by software, hardware, integrated circuits, firmware, micro code, and the like, operating alone or in combination. In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the manner in which the present embodiments are programmed.

In an embodiment, the processor 1330 implements one or more machine learning networks that are stored in the memory 1360. In general, a trained machine learning network mimics cognitive functions that humans associate with other human minds. In particular, by training based on training data the machine learning network is able to adapt to new circumstances and to detect and extrapolate patterns. Another term for “trained machine learning network” is “trained function”. In general, parameters of a machine learning network can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning networks can be adapted iteratively by several steps of training. In particular, within the training a certain cost function can be minimized. In particular, within the training of a neural network the backpropagation algorithm can be used. In particular, a machine learning network may comprise a neural network, a support vector machine, a decision tree and/or a Bayesian network, and/or the machine learning network can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network, and/or a generative adversarial network.

In an embodiment, the processor 1310 implements a diffusion process for training and configuring the model. The diffusion process consists of forward diffusion and reverse diffusion. Forward diffusion is used to add noise to the input image using a schedule which determines how much noise is added at the given step t. Reverse diffusion consists of multiple steps in which a small amount of noise is removed at every step. In an embodiment, the diffusion models use a modified U-Net architecture, for example as described above in FIG. 8. In an embodiment, the model(s) are provided by or implemented with a neural network trained using deep learning. The network(s) may be defined as a plurality of sequential feature units or layers. Sequential is used to indicate the general flow of output feature values from one layer to input to a next layer. The information from the next layer is fed to a next layer, and so on until the final output. The layers may only feed forward or may be bi-directional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction the input data. The features are learned to reconstruct lower level features (i.e., features at a more abstract or compressed level). For example, features for generating a fused image or higher resolution image are learned. For a next unit, features for reconstructing the features of the previous unit are learned, providing more abstraction. Each node of the unit represents a feature. Different units are provided for learning different features.

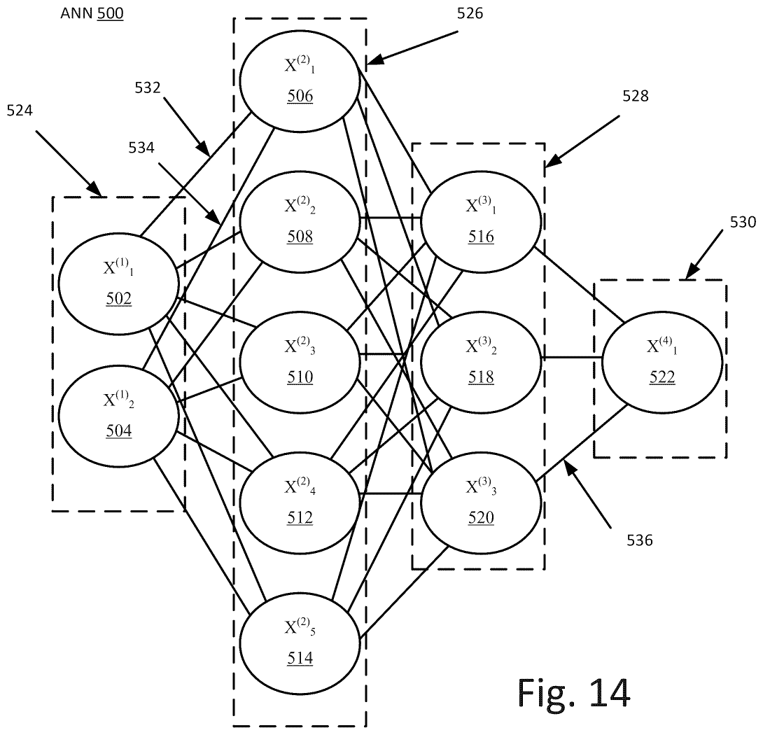

Various units or layers may be used, such as convolutional, pooling (e.g., max-pooling), deconvolutional, fully connected, or other types of layers. Within a unit or layer, any number of nodes is provided. For example, 100 nodes are provided. Later or subsequent units may have more, fewer, or the same number of nodes. In general, for convolution, subsequent units have more abstraction. FIG. 14 shows an embodiment of an artificial neural network (ANN) 500, in accordance with one or more embodiments. Alternative terms for “artificial neural network” are “neural network”, “artificial neural net” or “neural net”. The artificial neural network 500 may be used in part in, for example, the one or more machine learning based networks utilized for the first generative model 400 and/or second generative model 700, etc.

The artificial neural network 500 includes nodes 502-522 and edges 532, 534, . . . , 536, wherein each edge 532, 534, . . . , 536 is a directed connection from a first node 502-522 to a second node 502-522. In general, the first node 502-522 and the second node 502-522 are different nodes 502-522, it is also possible that the first node 502-522 and the second node 502-522 are identical. For example, in FIG. 14, the edge 532 is a directed connection from the node 502 to the node 506, and the edge 534 is a directed connection from the node 504 to the node 506. An edge 532, 534, . . . , 536 from a first node 502-522 to a second node 502-522 is also denoted as “ingoing edge” for the second node 502-522 and as “outgoing edge” for the first node 502-522.

In this embodiment, the nodes 502-522 of the artificial neural network 500 may be arranged in layers 524-530, wherein the layers may include an intrinsic order introduced by the edges 532, 534, . . . , 536 between the nodes 502-522. In particular, edges 532, 534, . . . , 536 may exist only between neighboring layers of nodes. In the embodiment shown in FIG. 5, there is an input layer 524 including only nodes 502 and 504 without an incoming edge, an output layer 530 including only node 522 without outgoing edges, and hidden layers 526, 528 in-between the input layer 524 and the output layer 530. In general, the number of hidden layers 526, 528 may be chosen arbitrarily. The number of nodes 502 and 504 within the input layer 524 usually relates to the number of input values of the neural network 500, and the number of nodes 522 within the output layer 530 usually relates to the number of output values of the neural network 500.

In particular, a (real) number may be assigned as a value to every node 502-522 of the neural network 500. Here, x⁽ⁿ⁾ᵢ denotes the value of the i-th node 502-522 of the n-th layer 524-530. The values of the nodes 502-522 of the input layer 524 are equivalent to the input values of the neural network 500, the value of the node 522 of the output layer 530 is equivalent to the output value of the neural network 500. Furthermore, each edge 532, 534, . . . , 536 may include a weight being a real number, in particular, the weight is a real number within the interval [−1, 1] or within the interval [0, 1]. Here, w^{(m,n)}_{i,j} denotes the weight of the edge between the i-th node 502-522 of the m-th layer 524-530 and the j-th node 502-522 of the n-th layer 524-530. Furthermore, the abbreviation w⁽ⁿ⁾_{i,j} is defined for the weight w^{(n,n+1)}_{i,j}.

In particular, to calculate the output values of the neural network 500, the input values are propagated through the neural network. In particular, the values of the nodes 502-522 of the (n+1)-th layer 524-530 may be calculated based on the values of the nodes 502-522 of the n-th layer 524-530 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 524 are given by the input of the neural network 500, wherein values of the first hidden layer 526 may be calculated based on the values of the input layer 524 of the neural network, wherein values of the second hidden layer 528 may be calculated based in the values of the first hidden layer 526, etc.

In order to set the values w^{(m,n)}_{i,j} for the edges, the neural network 500 has to be trained using training data. In particular, training data includes training input data and training output data (denoted as tᵢ). For a training step, the neural network 500 is applied to the training input data to generate calculated output data. In particular, the training data and the calculated output data include a number of values, said number being equal with the number of nodes of the output layer.

In particular, a comparison between the calculated output data and the training data is used to recursively adapt the weights within the neural network 500 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers δ⁽ⁿ⁾ⱼ may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δ⁽ⁿ⁺¹⁾ⱼ, if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 530, wherein f′ is the first derivative of the activation function, and y⁽ⁿ⁺¹⁾ⱼ is the comparison training value for the j-th node of the output layer 530.

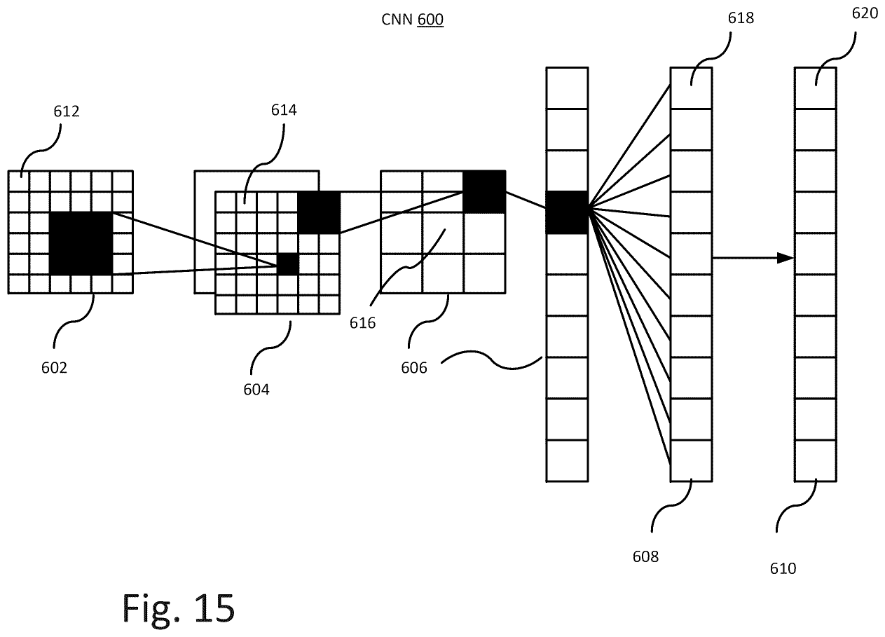

FIG. 15 shows a convolutional neural network (CNN) 600, in accordance with one or more embodiments. Machine learning networks described herein, such as, e.g., the first generative model 400 and/or second generative model 700 etc. may be implemented using convolutional neural network 600.

In the embodiment shown in FIG. 15 the convolutional neural network includes 600 an input layer 602, a convolutional layer 604, a pooling layer 606, a fully connected layer 608, and an output layer 610. Alternatively, the convolutional neural network 600 may include several convolutional layers 604, several pooling layers 606, and several fully connected layers 608, as well as other types of layers. The order of the layers may be chosen arbitrarily, usually fully connected layers 608 are used as the last layers before the output layer 610.

In particular, within a convolutional neural network 600, the nodes 612-620 of one layer 602-610 may be considered to be arranged as a d-dimensional matrix or as a d-dimensional image. In particular, in the two-dimensional case the value of the node 612-620 indexed with i and j in the n-th layer 602-610 may be denoted as x⁽ⁿ)_{[i,j]}. However, the arrangement of the nodes 612-620 of one layer 602-610 does not have an effect on the calculations executed within the convolutional neural network 600 as such, since these are given solely by the structure and the weights of the edges.

In particular, a convolutional layer 604 is characterized by the structure and the weights of the incoming edges forming a convolution operation based on a certain number of kernels. In particular, the structure and the weights of the incoming edges are chosen such that the values x⁽ⁿ⁾ₖ of the nodes 614 of the convolutional layer 604 are calculated as a convolution x⁽ⁿ⁾ₖ=Kₖ*x⁽ⁿ⁻¹⁾ based on the values x⁽ⁿ⁻¹⁾ of the nodes 612 of the preceding layer 602, where the convolution * is defined in the two-dimensional case as:

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kk is a d-dimensional matrix (in this embodiment a two-dimensional matrix), which is usually small compared to the number of nodes 612-618 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 612-620 in the respective layer 602-610. In particular, for a convolutional layer 604, the number of nodes 614 in the convolutional layer is equivalent to the number of nodes 612 in the preceding layer 602 multiplied with the number of kernels.

If the nodes 612 of the preceding layer 602 are arranged as a d-dimensional matrix, using a plurality of kernels may be interpreted as adding a further dimension (denoted as “depth” dimension), so that the nodes 614 of the convolutional layer 604 are arranged as a (d+1)-dimensional matrix. If the nodes 612 of the preceding layer 602 are already arranged as a (d+1)-dimensional matrix including a depth dimension, using a plurality of kernels may be interpreted as expanding along the depth dimension, so that the nodes 614 of the convolutional layer 604 are arranged also as a (d+1)-dimensional matrix, wherein the size of the (d+1)-dimensional matrix with respect to the depth dimension is by a factor of the number of kernels larger than in the preceding layer 602.

The advantage of using convolutional layers 604 is that spatially local correlation of the input data may exploited by enforcing a local connectivity pattern between nodes of adjacent layers, in particular by each node being connected to only a small region of the nodes of the preceding layer.

In embodiment shown in FIG. 15, the input layer 602 includes 36 nodes 612, arranged as a two-dimensional 6×6 matrix. The convolutional layer 604 includes 72 nodes 614, arranged as two two-dimensional 6×6 matrices, each of the two matrices being the result of a convolution of the values of the input layer with a kernel. Equivalently, the nodes 614 of the convolutional layer 604 may be interpreted as arranges as a three-dimensional 6×6×2 matrix, wherein the last dimension is the depth dimension.

A pooling layer 606 may be characterized by the structure and the weights of the incoming edges and the activation function of its nodes 616 forming a pooling operation based on a non-linear pooling function f. For example, in the two dimensional case the values x⁽ⁿ⁾ of the nodes 616 of the pooling layer 606 may be calculated based on the values x⁽ⁿ⁻¹⁾ of the nodes 614 of the preceding layer 604 as

$${x}^{( n )} [ i , j ] = f ( {x}^{( n - 1 )} [ {id}_{1} , {jd}_{2} ] , … , {x}^{( n - 1 )} [ {id}_{1} + {d}_{1} - 1 , {jd}_{2} + {d}_{2} - 1 ] )$$

In other words, by using a pooling layer 606, the number of nodes 614, 616 may be reduced, by replacing a number d1·d2 of neighboring nodes 614 in the preceding layer 604 with a single node 616 being calculated as a function of the values of said number of neighboring nodes in the pooling layer. In particular, the pooling function f may be the max-function, the average, or the L2-Norm. In particular, for a pooling layer 606 the weights of the incoming edges are fixed and are not modified by training.

The advantage of using a pooling layer 606 is that the number of nodes 614, 616 and the number of parameters is reduced. This leads to the amount of computation in the network being reduced and to a control of overfitting.

In the embodiment shown in FIG. 15, the pooling layer 606 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 608 may be characterized by the fact that a majority, in particular, all edges between nodes 616 of the previous layer 606 and the nodes 618 of the fully-connected layer 608 are present, and wherein the weight of each of the edges may be adjusted individually.

In this embodiment, the nodes 616 of the preceding layer 606 of the fully-connected layer 608 are displayed both as two-dimensional matrices, and additionally as non-related nodes (indicated as a line of nodes, wherein the number of nodes was reduced for a better presentability). In this embodiment, the number of nodes 618 in the fully connected layer 608 is equal to the number of nodes 616 in the preceding layer 606. Alternatively, the number of nodes 616, 618 may differ.

A convolutional neural network 600 may also include a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks may be wired using summation (residual/dense neural networks), element-wise multiplication (attention) or other differentiable operators. Therefore, the convolutional neural network architecture may be nested rather than being sequential if the whole pipeline is differentiable.

In particular, convolutional neural networks 600 may be trained based on the backpropagation algorithm. For preventing overfitting, methods of regularization may be used, e.g. dropout of nodes 612-620, stochastic pooling, use of artificial data, weight decay based on the L1 or the L2 norm, or max norm constraints. Different loss functions may be combined for training the same neural network to reflect the joint training objectives. A subset of the neural network parameters may be excluded from optimization to retain the weights pretrained on another datasets.

The operator interface 140 includes an input device and an output device. The input may be an interface, such as interfacing with a computer network, memory, database, medical image storage, or other source of input data. The input may be a user input device, such as a mouse, trackpad, keyboard, roller ball, touch pad, touch screen, or another apparatus for receiving user input. The output is a display device but may be an interface. The reconstructed images and/or quantitative maps are displayed. For example, a high resolution image of a region of the patient 225 is displayed. The display is a CRT, LCD, plasma, projector, printer, or other display device. The display is configured by loading an image to a display plane or buffer. The display is configured to display the image of the region of the patient 225. The operator interface may include a graphical user interface (GUI) enabling user interaction with the medical imaging device 105 and enables user modification or selections in substantially real time.

While the invention has been described above by reference to various embodiments, many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

The following is a list of non-limiting illustrative embodiments disclosed herein:

Illustrative embodiment 1: a method for quantitative MRI using Generative AI, the method comprising: training a first generative model to reconstruct an MR image from acquired MR data; and training a second generative model to generate quantitative map data from the acquired MR data, wherein a data consistency term derived from the acquired MR data is used for regularization of the second generative model; and storing the first generative model and second generative model.

Illustrative embodiment 2: the method of Illustrative embodiment 1, wherein the first generative model and second generative model comprise diffusion models, wherein training comprises a forward process and an inference stage for each of training the first generative model and the second generative model.

Illustrative embodiment 3: the method of illustrative embodiment 2, wherein the first generative model and the second generative model are trained and used for inference separately.

Illustrative embodiment 4: the method of illustrative embodiment 2, wherein the first generative model and the second generative model are separately trained but include joint consideration during inference.

Illustrative embodiment 5: the method of illustrative embodiment 2, wherein the first generative model and the second generative model are jointly trained and used for inference.

Illustrative embodiment 6: the method of illustrative embodiment 2, wherein one or more measurement values are used for regularization during an inference stage of the first generative model.

Illustrative embodiment 7: the method of illustrative embodiment 1, wherein the first generative model and second generative model comprise at least one of an auto encoder, a variational auto encoder, a denoising auto encoder, a restricted boltzmann machine, a generative adversarial network, a denoising diffusion probabilistic model, a score-based diffusion model, a poisson flow generative model, flow matching, rectified flow, or auto regressive model.

Illustrative embodiment 8: the method of illustrative embodiment 1, wherein the quantitative map data comprises an ADC value, wherein the data consistency term comprises B-values from the acquired MR data.

Illustrative embodiment 9: the method of illustrative embodiment 1, wherein the quantitative map data comprises at least one of the following: diffusion-related parameters, ADC, tensor parameters, IVIM parameters, Kurtosis parameters, T1, T2, T2*, T1r, tissue fat/iron, Volumetry, Perfusion, blood flow, blood volume, time-to-peak, mean transit time, flow, tissue viscoelastic properties (elastography), dynamic contrast enhancement, quantitative susceptibility mapping, chemical exchange saturation transfer, Magnetization transfer/transfer ratio, spectroscopy, or temperature mapping.

Illustrative embodiment 10: the method of illustrative embodiment 1, further comprising: acquiring the MR data; applying the first generative model and second generative model to the MR data; and outputting a reconstructed MR image and quantitative map data.

Illustrative embodiment 11: the method of illustrative embodiment 10, further comprising: displaying the reconstructed MR image and quantitative map data.

Illustrative embodiment 12: a method for quantitative MRI, the method comprising: acquiring MR imaging data; inputting the MR imaging data into a first generative model trained to reconstruct an image and a second generative model trained to generate quantitative MRI data, wherein the first generative model is constrained by a data consistency term based on measurement data, wherein the second generative model is regularized by a constrained mathematical model fit; and outputting the reconstructed image and the quantitative MRI data.

Illustrative embodiment 13: the method of illustrative embodiment 12, wherein the first generative model and the second generative model are trained and used for inference separately.

Illustrative embodiment 14: the method of illustrative embodiment 12, wherein the wherein the first generative model and the second generative model are separately trained but include joint consideration during inference.

Illustrative embodiment 15: the method of illustrative embodiment 12, wherein the first generative model and the second generative model are jointly trained and used for inference.

Illustrative embodiment 16: the method of illustrative embodiment 12, wherein the quantitative MRI data comprises at least one of the following: diffusion-related parameters, ADC, tensor parameters, IVIM parameters, Kurtosis parameters, T1, T2, T2*, T1r, Muscle fat/iron, liver fat/iron, Volumetry, Perfusion, blood flow, blood volume, time-to-peak, mean transit time, flow, tissue viscoelastic properties (elastography), dynamic contrast enhancement, quantitative susceptibility mapping, chemical exchange saturation transfer, Magnetization transfer/transfer ratio, spectroscopy, or temperature mapping.

Illustrative embodiment 17: the method of illustrative embodiment 12, wherein the first generative model and second generative model comprise at least one of an auto encoder, a variational auto encoder, a denoising auto encoder, a restricted boltzmann machine, a generative adversarial network, a denoising diffusion probabilistic model, a score-based diffusion model, a poisson flow generative model, flow matching, rectified flow, or auto regressive model.

Illustrative embodiment 18: a system for quantitative MRI, the system comprising: a medical imaging device configured to acquire MR data; a memory configured to store a first generative model configured to reconstruct an MR image from the MR data and a second generative model trained to learn a probability density of quantitative map data and generate quantitative map data wherein the quantitative map data generation is constrained by an exponential fit provided by a priori probability density function from the first generative model; and a processor configured to reconstruct an MR image using the first generative model and generate a quantitative map using the second generative model.

Illustrative embodiment 19: the system of illustrative embodiment 18, wherein the first generative model and the second generative model are jointly trained and used for inference.

Illustrative embodiment 20: the system of illustrative embodiment 18, further comprising: a display configured to display the MR image and the quantitative map.

1. A method for quantitative MRI using Generative AI, the method comprising:
training a first generative model to reconstruct an MR image from acquired MR data;
training a second generative model to generate quantitative map data from the acquired MR data, wherein a data consistency term derived from the acquired MR data is used for regularization of the second generative model; and
storing the first generative model and second generative model.

2. The method of claim 1, wherein the first generative model and second generative model comprise diffusion models, wherein training comprises a forward process and an inference stage for each of training the first generative model and the second generative model.

3. The method of claim 2, wherein the first generative model and the second generative model are trained and used for inference separately.

4. The method of claim 2, wherein the first generative model and the second generative model are separately trained but include joint consideration during inference.

5. The method of claim 2, wherein the first generative model and the second generative model are jointly trained and used for inference.

6. The method of claim 2, wherein one or more measurement values are used for regularization during an inference stage of the first generative model.

7. The method of claim 1, wherein the first generative model and second generative model comprise at least one of an auto encoder, a variational auto encoder, a denoising auto encoder, a restricted boltzmann machine, a generative adversarial network, a denoising diffusion probabilistic model, a score-based diffusion model, a poisson flow generative model, flow matching, rectified flow, or auto regressive model.

8. The method of claim 1, wherein the quantitative map data comprises an ADC value, wherein the data consistency term comprises B-values from the acquired MR data.

9. The method of claim 1, wherein the quantitative map data comprises at least one of the following: diffusion-related parameters, ADC, tensor parameters, IVIM parameters, Kurtosis parameters, T1, T2, T2*, T1r, tissue fat/iron, Volumetry, Perfusion, blood flow, blood volume, time-to-peak, mean transit time, flow, tissue viscoelastic properties (elastography), dynamic contrast enhancement, quantitative susceptibility mapping, chemical exchange saturation transfer, Magnetization transfer/transfer ratio, spectroscopy, or temperature mapping.

10. The method of claim 1, further comprising:
acquiring the MR data;
applying the first generative model and second generative model to the MR data; and
outputting a reconstructed MR image and quantitative map data.

11. The method of claim 10, further comprising:
displaying the reconstructed MR image and quantitative map data.

12. A method for quantitative MRI, the method comprising:
acquiring MR imaging data;
inputting the MR imaging data into a first generative model trained to reconstruct an image and a second generative model trained to generate quantitative MRI data, wherein the first generative model is constrained by a data consistency term based on measurement data, wherein the second generative model is regularized by a constrained mathematical model fit; and
outputting the reconstructed image and the quantitative MRI data.

13. The method of claim 12, wherein the first generative model and the second generative model are trained and used for inference separately.

14. The method of claim 12, wherein the wherein the first generative model and the second generative model are separately trained but include joint consideration during inference.

15. The method of claim 12, wherein the first generative model and the second generative model are jointly trained and used for inference.

16. The method of claim 12, wherein the quantitative MRI data comprises at least one of the following: diffusion-related parameters, ADC, tensor parameters, IVIM parameters, Kurtosis parameters, T1, T2, T2*, T1r, Muscle fat/iron, liver fat/iron, Volumetry, Perfusion, blood flow, blood volume, time-to-peak, mean transit time, flow, tissue viscoelastic properties (elastography), dynamic contrast enhancement, quantitative susceptibility mapping, chemical exchange saturation transfer, Magnetization transfer/transfer ratio, spectroscopy, or temperature mapping.

17. The method of claim 12, wherein the first generative model and second generative model comprise at least one of an auto encoder, a variational auto encoder, a denoising auto encoder, a restricted boltzmann machine, a generative adversarial network, a denoising diffusion probabilistic model, a score-based diffusion model, a poisson flow generative model, flow matching, rectified flow, or auto regressive model.

18. A system for quantitative MRI, the system comprising:
a medical imaging device configured to acquire MR data;
a memory configured to store a first generative model configured to reconstruct an MR image from the MR data and a second generative model trained to learn a probability density of quantitative map data and generate quantitative map data wherein the quantitative map data generation is constrained by an exponential fit provided by a priori probability density function from the first generative model; and
a processor configured to reconstruct an MR image using the first generative model and generate a quantitative map using the second generative model.

19. The system of claim 18, wherein the first generative model and the second generative model are jointly trained and used for inference.

20. The system of claim 18, further comprising:
a display configured to display the MR image and the quantitative map.
